(19) United States
(12) Patent Application Publication
(10) Pub. No.: US 2012/0225947 A1
(43) Pub. Date: Sep. 6, 2012

(54) RETINAL NEUROPROTECTION BY ION CHANNEL BLOCKERS REGULATED BY THE SUR SUBUNIT

(75) Inventors: Michel Polak, Paris (FR); Marianne Berdugo Polak, Paris (FR); Francine Behar-Cohen, Paris (FR); Patricia Crisanti Lassiaz, Paris (FR)

(73) Assignee: ASSISTANCE PUBLIQUE - HOPITAUX DE PARIS, PARIS (FR)

(21) Appl. No.: 13/497,938

(22) PCT Filed: Sep. 23, 2010
(86) PCT No.: PCT/EP2010/064040

§ 371 Date: Mar. 23, 2012

(30) Foreign Application Priority Data
Sep. 24, 2009 (EP) 09305892.3

(51) Int. Cl. A61K 31/18; A61P 27/06; A61P 27/10; A61P 27/02

(52) U.S. Cl. 514/593

(57) ABSTRACT

The present invention relates to the use of blockers of ion channels regulated by the SUR subunit, for the treatment and/or prevention of eye diseases associated with ischemia and/or retinal excitotoxicity.

The invention related to the field of medicine, and more particularly to that of the treatment of retinal diseases.

### TECHNOLOGICAL BACKGROUND OF THE INVENTION

The retina is a membrane approximately 0.5 mm thick lining the inner surface of the eye. Light that strikes the retina is converted to an electrical signal through the process of phototransduction which takes place in retinal photoreceptors. This electrical signal is then transmitted to the brain via retinal ganglion cells and bipolar cells, then along the optic nerve. From an embryologic and histologic standpoint, the retina is an extension of the central nervous system because it forms during embryogenesis from lateral outgrowths of the neural ampulla forming optic vesicles which later give rise to the retina. Thus, the eye, and more particularly the retina, is directly connected to the brain by the optic nerve. The latter is formed by axons of retinal ganglion cells which transmit the information received by the retina along the optic tracts to the occipital cortex where the information is processed.

Clinically, the retina is the site of many diseases including those associated with retinal ischemia which results in retinal cell degeneration leading to visual impairment. These ischemic disorders can be observed in various acute optic neuropathies and retinopathies such as retinal artery or vein occlusions or eye injuries, or in chronic diseases such as age-related macular degeneration (ARMD), glaucoma, diabetic retinopathy or else retinopathy of prematurity. Glaucoma and ARMD are the two leading causes of blindness in industrialized countries and constitute a major public health concern.

Many studies have shown that excitotoxicity phenomena were partly responsible for ischemic retinal neuropathies, particularly in the case of glaucoma (Franzco, 2006). This is because ischemic insult results in hyperstimulation of glutamate receptors, an excitatory neurotransmitter. This activation induces calcium overload which causes cell damage and death. The involvement of glutamate ionotropic receptors in ischemic toxicity was confirmed by the fact that their antagonists exert a neuroprotective effect (Lam et al., 1997; Yoon et al., 1989; Osborne et al., 1996; Tsukahara et al., 1992). Excitotoxicity may also be responsible for retinal cell degeneration in pathologies such as retinal detachment (Sherry and Townes-Anderson, 2000), in retinal damage related to diseases involving vascular dysfunction such as sickle cell retinopathy, or else in the case of retinal laser surgery (photocoagulation).

To date, many molecules have been tested in the treatment or prevention of pathologies related to retinal ischemia or retinal excitotoxicity, such as, for example, competitive and noncompetitive glutamate receptor antagonists (Lam et al., 1997; Yoon et al., 1989; Vorwerck et al., 1996); free radical scavengers (Celeci et al., 2002; Szabo et al., 2001); adrenoreceptor antagonists (Donello et al., 2001; Chao et al., 2001); neurotrophic factors (Fontaine et al., 2002) or else inhibitors of presynaptic glutamate release.

However, the method of administration of some of these molecules, and their activity on other organs, may cause undesirable side effects. Moreover, their efficacy at clinically feasible doses still remains very insufficient. Thus there continues to be a real need to find novel compounds for the treatment and/or prevention of these diseases.

### SUMMARY OF THE INVENTION

The object of the present invention is to provide novel compounds that can be used for the treatment and/or prevention of diseases associated with retinal ischemia and/or retinal excitotoxicity.

The present invention relates first of all to a blocker of ion channels regulated by the SUR (SulfonylUrea Receptor) subunit for a use in the treatment and/or prevention of a disease associated with retinal ischemia and/or retinal excitotoxicity.

According to one embodiment, the blocker is selected from the group consisting of a sulfonylurea and a meglitinide, and a combination thereof. In particular, the blocker is selected from the group consisting of glibenclamide, acetohexamide, carbutamide, glibornuride, chlorpropamide, tolbutamide, tolazamide, glipizide, gliclazide, gliquidone, glyclopyramide, glisoxepide, glimepiride, repaglinide, nateglinide and mitiglinide, and any combination thereof. Preferably, the blocker is glibenclamide.

According to a particular embodiment, the blocker is administered in combination with another active substance, simultaneously or sequentially.

According to one embodiment, the disease associated with retinal ischemia and/or retinal excitotoxicity is selected from the group consisting of glaucoma, glaucomatous optic neuropathies without hypertonia, age-related macular degeneration, acute or chronic intraocular inflammations (uveitis, uveoretinitis, choroiditis), ischemic or toxic optic neuropathies, endophthalmitis, infectious retinitis, diabetic retinopathy, retinopathy of prematurity, ischemic retinitis proliferans, retinitis pigmentosa, hemoglobinopathy retinopathies, photodegeneration, retinal detachment, retinal and choroidal vascular disorders (stenosis, thrombosis and vascular occlusions), retinal and/or choroidal hemorrhage, and hereditary or acquired retinal degeneration. Preferably, the disease associated with retinal ischemia and/or retinal excitotoxicity is selected from the group consisting of glaucoma, age-related macular degeneration, diabetic retinopathy, retinopathy of prematurity, ischemic retinitis proliferans, and retinitis pigmentosa.

According to a particular embodiment, the subject to be treated is a human being, in particular an infant, a child or an adult. According to another embodiment the subject to be treated is an animal selected from the group consisting of a dog, a cat, a horse, a cow, a sheep, a pig and a non-human primate.

According to another particular embodiment, the blocker is administered by the intravitreal route, by the subconjunctival route, by the oral route, by topical instillation, by the periocular or intraocular route or by the parenteral route. Preferably, the blocker is administered by the intravitreal route, by the subconjunctival route, by the oral route or by topical instillation.

In a second aspect, the present invention relates to the use of a blocker of ion channels regulated by the SUR subunit for producing a medicament intended for the treatment and/or prevention of an eye disease associated with retinal ischemia and/or retinal excitotoxicity.

In another aspect, the present invention relates to a method of treatment and/or prevention of an eye disease associated with retinal ischemia and/or retinal excitotoxicity in a subject, said method comprising administering an effective therapeutic dose of a blocker of ion channels regulated by the SUR subunit to said subject.

### BRIEF DESCRIPTION OF DRAWINGS

FIG. 1: Negative control without TUNEL enzyme on a control retinal slice. (A) DAPI labeling of retinal slice. (B) TUNEL labeling of retinal slice. (C) Photos A and B superimposed.

FIG. 2: Positive control carried out by examining superficial corneal epithelium of the retinas studied, which normally contain apoptotic cells. (A) DAPI labeling. (B) TUNEL labeling. (C) Photos A and B superimposed.

FIG. 3: NMDA-induced retinal cell degeneration: bipolar and ganglion cells layers. (A) DAPI labeling. (B) TUNEL labeling. (C) Photos A and B superimposed.

FIG. 4: Effect of DMSO on retinal cells with and without NMDA injection. (A) Cell labeling with DAPI and TUNEL after DMSO injection. (B) Negative control without TUNEL enzyme. (C) DAPI and TUNEL labeling of retinal cells after DMSO injection with NMDA. (D) DAPI and TUNEL labeling of retinal cells after NMDA injection only.

FIG. 5: Histogram of mean number of apoptotic cells per field for each group of rats. DMSO: DMSO injection only (group 5); NMDA+DMSO: co-injection of NMDA and DMSO (group 6); NMDA: NMDA injection only (group 4); GLI 100 ng: co-injection of NMDA and 100 ng glibenclamide (group 3); GLI 10 ng: co-injection of NMDA and 10 ng glibenclamide (group 2); GLI 1 ng: co-injection of NMDA and 1 ng glibenclamide (group 1).

FIG. 6: Graph illustrating number of apoptotic cells per field of retinal section after intravitreal injection of 1 ng glibenclamide (gli 1 ng IVT), 0.01 ng glibenclamide (gli 0.01 ng IVT) and DMSO solution (DMSO IVT). Nonparametric Mann-Whitney test (*P<0.05;**P<0.01;***P<0.001).

FIG. 7: Structure of retinal layers labeled with DAPI. (A) after NMDA and DMSO co-injection; (B) after NMDA and 1 ng glibenclamide co-injection; (C) after NMDA and 0.01 ng glibenclamide co-injection.

FIG. 8: Graph illustrating number of apoptotic cells per field of retinal section after oral administration of glibenclamide 10 mg/kg (gli 10 mg/kg per os) or DMSO solution (DMSO per os) and intravitreal injection of NMDA the same day. Nonparametric Mann-Whitney test (*P=0.012).

FIG. 9: Graph illustrating number of apoptotic cells per field of retinal section after subconjunctival administration of 1 ng glibenclamide (gli 1 ng SCJ) or DMSO solution (DMSO SCJ) and intravitreal injection of NMDA the same day. Nonparametric Mann-Whitney test (***P=0.0002).

FIG. 10: Graph illustrating number of apoptotic cells per field of retinal section after intravitreal administration of 100 μg (gli 100 micro D-5) or 100 ng (gli 100 ng D-5) glibenclamide or BSS solution (BSS D-5) 5 days before intravitreal NMDA injection. Nonparametric Mann-Whitney test (*** P<0.0001).

### DETAILED DESCRIPTION OF THE INVENTION

ATP-dependent potassium channels (K(ATP)) are hetero-octomers composed of four Kir6.x (inward rectifier K⁺ channel) subunits which form the ion channel itself, and four regulatory subunits, the SUR (sulfonylurea receptors). These channels are present in many cell types, particularly pancreatic cells and neuronal cells, in the form Kir6.x/SURy.

In pancreatic cells, Kir6.2/SUR1 channels are involved in glucose regulation. Hyperglycemia induces an increase in glucose metabolism by pancreatic beta cells, leading to increased ATP levels and a decrease in the ADP/ATP ratio in the cell. This decreased ratio causes the K(ATP) Kir6.2/SUR1 channels to close, resulting in depolarization of the cell. This induces opening of voltage-gated calcium channels, increased intracellular calcium levels and finally insulin release.

Sulfonylureas and meglitinides are hypoglycemic drugs used in the treatment of diabetes. They bind to the SUR subunit of Kir6.2/SUR1 channels and block their opening, thereby promoting increased release of insulin.

Kir6.x/SURy channels are also present in brain (Liss et al., 2001) where they play a role in protecting the central nervous system (CNS) from ischemia. In fact, activation of these channels is induced during a metabolic state of ATP depletion, such as hypoxia, and permits a reduction in energy consumption. However, the involvement of these channels in cerebral edema secondary to ischemic insult has also been demonstrated and the use of sulfonylureas has been shown to exert a neuroprotective effect in an in vitro model of brain ischemia (Nistico et al., 2007).

It has also been shown that the regulatory SURI subunit is overexpressed in CNS lesions, particularly in brain ischemia. This overexpression is correlated with the expression of the ATP-dependent nonselective calcium cation channel (NC_{Ca}-ATP) of which SUR 1 is also the regulatory subunit. On the other hand, it does not appear to be correlated with expression of the Kir6.2/SUR1 channel (Simard et al., 2006). The NC_{Ca}-ATP channel is not constitutively expressed in the CNS but only during neuronal damage or ischemic episodes. Administration of sulfonylureas during brain ischemia, by virtue of their binding to SUR1, cuts patient mortality in half and also reduces the extent of damage (Simard et al., 2008).

On the other hand, the neuroprotective effect of sulfonylureas as ion channel blockers has never been demonstrated in tissues other than the central nervous system.

In the present application the inventors have demonstrated that administration of a blocker of ion channels regulated by the SUR subunit enables the treatment or prevention of retinal pathologies associated with ischemia or with retinal excitotoxicity phenomena or with myopia. In particular, the inventors have shown, in an in vivo model of retinal degeneration, that said blockers have a retinal neuroprotective effect and that their use decreases retinal damage induced by administration of an excitotoxic compound such as N-methyl-D-asparatate (NMDA). The inventors have further shown that said blockers may be efficiently administered by various routes, in particular by the intravitreal, oral or subconjunctival route, and that they are totally devoid of toxicity to retinal cells, and this, even at high doses.

In a first aspect, the present invention relates to a blocker of ion channels regulated by the SUR subunit for a use in the treatment and/or prevention of an eye disease associated with retinal ischemia and/or retinal excitotoxicity.

In the present document, the phrase “a blocker of ion channels regulated by the SUR subunit” refers to a compound able to block the opening of ion channels regulated by the SUR subunit and thereby able to block ion flux through the cell membrane, by binding to a subunit of said channels. Ion channels regulated by the SUR subunit comprise, for example, potassium channels Kir6.2/SUR1, nonselective ATP-dependent calcium cation channels (NC_{Ca}-ATP), and any channel permitting ion conductance, coupled to the SUR1 or SUR2 subunit (A or B).

According to one embodiment, the blocker of ion channels regulated by the SUR subunit is a ligand of the SUR subunit. Preferably, the SUR subunit is selected from the group consisting of SUR1 (GeneID: 6833), SUR2A and SUR2B (GeneID: 10060; both encoded by the gene ABCC9 (ATP-binding cassette, sub-family C (CFTR/MRP), member 9, also known as ABC37, CMDIO, FLJ36852, SUR2) and derived by alternative splicing). In a particularly preferred manner, the SUR subunit is the SUR1 subunit.

According to one embodiment of the invention, the blocker of ion channels regulated by the SUR subunit is selected from the group consisting of sulfonylureas and meglitinides (or glinides), and any combination thereof.

The sulfonylureas which may be used according to the invention comprise, by way of example and not by way of limitation, glibenclamide (or glyburide), acetohexamide, carbutamide, glibornuride, chlorpropamide, tolbutamide, tolazamide, glipizide, gliclazide, gliquidone, glyclopyramide, glisoxepide and glimepiride.

The meglitinides which may be used according to the invention comprise, by way of example and not by way of limitation, repaglinide, nateglinide and mitiglinide.

According to a particular embodiment, the blocker of ion channels regulated by the SUR subunit is selected from the group consisting of glibenclamide, acetohexamide, carbutamide, glibornuride, chlorpropamide, tolbutamide, tolazamide, glipizide, gliquidone, glyclopyramide, glisoxepide, glimepiride, repaglinide, nateglinide and mitiglinide, and any combination thereof.

According to a preferred embodiment, the blocker of ion channels regulated by the SUR subunit is a sulfonylurea, preferably selected from the group consisting of glibenclamide, acetohexamide, carbutamide, glibornuride, chlorpropamide, tolbutamide, tolazamide, glipizide, gliquidone, glyclopyramide, glisoxepide and glimepiride. In a particularly preferred manner, the blocker of ion channels regulated by the SUR subunit is glibenclamide.

The blocker of ion channels regulated by the SUR subunit can be used alone or in combination with one or more other blockers of said channels.

The blocker of ion channels regulated by the SUR subunit can also be used in combination with one or more other active substances. The blocker and said active substance(s) can be administered simultaneously or sequentially. Said substances can be chosen in particular so as to suppress or limit the hypoglycemia induced by systemic administration of hypoglycemic agents such as sulfonylureas or meglitinides, such as for example, glucose or glucagon. They can also be chosen so as to enhance the neuroprotective effect of the administered composition. In such case, said substances can be selected from among the different substances known to have neuroprotective activity such as, for example, dizocilpine, memantine, riluzole, amantadine, dextromethorphan, dextrorphan, lacosamide, aptiganel, brimonidine and other α-2adrenergic agonists, diltiazem, sirtuins and the activators thereof such as resverastrol, rapamycin and analogs, dopaminergic agents, steroidal and nonsteroidal antiinflammatory agents, in particular acetylsalicylic acid, so-called “insulin-like” growth factors, in particular insulin, IGF-1, IGF-2, growth hormone and analogs thereof, estrogen and progesterone and analogs thereof, statins, neurotrophic factors in particular NGF, PEDF, GDNF, CNTF, VEGF, the analogs and activators thereof, antioxidant free radical scavengers, or else stem cells and bone marrow stromal cells. Alternatively, said substances can be selected from among the different substances known to have anti-ischemic or anti-excitotoxic properties such as, for example, dizocilpine (MK 801), magnesium, dextromethorphan, flupirtine, memantine, ketamine, SOD/catalase, lazaroid, dimethylthiourea, diosmin/hesperidin, melatonin, vitamin E, lipoic acid, deferoxamine, ibuprofen, L-NMMA, L-NNA, aminoguanidine, L-NAME, flunarizine, ganglioside, betaxolol, genistein and ifenprodil.

The blocker according to the invention can also be administered in combination with one or more substances selected from the group consisting of beta-blockers (for example timolol), alpha-2 adrenergic agonists (for example brimonidine), prostaglandin F2 alpha analogs (for example latanoprost), prostamides (for example bimatoprost), carbonic anhydrase inhibitors by the local and/or systemic route (for example dorzolamine or acetazolamide), epinephrine and epinephrine compounds, antimetabolites such as mitomycin C, 5-fluorouracil or 5-chlorouracil, thalidomide, inhibitors of growth factors FGF, VEGF, PGF or their receptors, inhibitors of IGF (insulin-like growth factor) of IRS-1 or IRS-2, metalloproteinase inhibitors, corticosteroids or derivatives, nonsteroidal anti-inflammatories, αvβ3 and αvβ5 integrin inhibitors, angiopoietin inhibitors, statins (for example endostatin, angiostatin, octreotide), carboxyamidotriazole, estradiol and derivatives, thrombospondins, parasympathicolytic agents, sympathomimetic agents, sulfadiazine, pyrimethamine, folinic acid, antiinfective or antiparasitic agents (for example hydroxynaphthoquinones), vancomycin, amikacin, ceftazidime, gentamicin, angiotensin converting enzyme inhibitors (for example candesartan) or inhibitors or activators of protein kinase C, carbogen and tissue plasminogen activator.

The blocker according to the invention can also be administered in combination with one or more substances used to treat ocular hypertension such as beta-blockers (for example timolol), alpha-2 adrenergic agonists (for example brimonidine), prostaglandin F2 alpha analogs (for example latanoprost), prostamides (for example bimatoprost), carbonic anhydrase inhibitors by the local and/or systemic route (for example dorzolamine or acetazolamide), epinephrine and epinephrine compounds. The blocker according to the invention can also be administered to patients treated for ocular hypertension by surgical means such as trabeculectomy or deep sclerectomy, laser trabeculoplasty, ultrasound treatment or cryosurgery. The blocker according to the invention can also be administered in combination with antimetabolites such as mitomycin C, 5-fluorouracil or 5-chlorouracil.

The blocker according to the invention can also be administered in combination with one or more substances used to treat or to inhibit neovascularization such as thalidomide, inhibitors of growth factors FGF, VEGF, PGF or their receptors, inhibitors of IGF (insulin-like growth factor) of IRS-1 or IRS-2, metalloproteinase inhibitors, corticosteroids and derivatives, nonsteroidal anti-inflammatories, αvβ3 and αvβ5 integrin inhibitors, angiopoietin inhibitors, statins (for example endostatin, angiostatin, octreotide), carboxyamidotriazole, estradiol and derivatives, and thrombospondins. The blocker according to the invention can also be administered to patients undergoing surgical treatment to inhibit neovascularization, such as laser treatment, dynamic phototherapy, surgery, radiotherapy or transpupillary thermotherapy.

The blocker according to the invention can also be used in the treatment of uveitis in combination with one or more substances used to treat uveitis. In particular, it can be used in combination with corticosteroids, nonsteroidal anti-inflammatories, parasympathicolytic agents and sympathomimetic agents.

The blocker according to the invention can also be used in the treatment of retinochoroiditis in combination with one or more substances used to treat these diseases such as corticosteroids, sulfadiazine, pyrimethamine, folinic acid, antiinfective or antiparasitic agents (for example hydroxynaphthoquinones).

The blocker according to the invention can also be used in the treatment of endophthalmitis in combination with one or more substances used to treat this disease such as vancomycin, amikacin, ceftazidime, gentamicin or corticosteroids. It can also be administered to patients undergoing vitrectomy.

The blocker according to the invention can also be administered to patients undergoing surgical treatment to treat diabetic retinopathy such as treatment of neovascularizations, laser photocoagulation of diabetic macular edema, vitrectomy or intravitreal corticosteroid implants. It can also be used in combination with one or more substances used to treat diabetic retinopathy such as for example angiotensin converting enzyme inhibitors (for example candesartan) or inhibitors or activators of protein kinase C.

The blocker according to the invention can also be administered to patients undergoing surgical treatment to treat retinopathy of prematurity such as laser treatment, cryotherapy, vitrectomy or scleral buckling. It can also be used in combination with one or more substances used to treat retinopathy of prematurity such as for example nonsteroidal antiinflammatory agents.

The blocker according to the invention can also be administered to patients undergoing surgical treatment to treat retinal stenosis, occlusion and thrombosis such as anterior chamber paracentesis, carotid endarterectomy or treatment of neovascularizations. It can also be used in combination with one or more substances used to treat stenosis, occlusion or thrombosis such as corticosteroids or carbogen.

The blocker according to the invention can also be administered to patients undergoing surgical treatment to treat retinochoroidal hemorrhage such as injection of gas tamponade (for example perfluorocarbon). It can also be used in combination with one or more substances used to treat retinochoroidal hemorrhage such as tissue plasminogen activator (tPA).

The blocker according to the invention can also be used in combination with gene therapy of retinitis pigmentosa.

When the blocker of the invention is administered to patients undergoing a surgical treatment, it can be administered before, during or after the surgical treatment.

In the spirit of the present document, the term “treatment” denotes any act by which to reduce, eliminate or delay the symptoms associated with a disease. The term “prevention” denotes any action intended to prevent the appearance of the symptoms associated with a disease. More specifically, the phrase “treatment of an eye disease associated with retinal ischemia and/or retinal excitotoxicity” refers to any act by which to reduce, eliminate or delay the degeneration of retinal cells. The phrase “prevention of an eye disease associated with retinal ischemia and/or retinal excitotoxicity” refers to any action intended to prevent the development of the phenomenon of retinal cell degeneration.

The phrase “eye disease associated with retinal ischemia and/or retinal excitotoxicity”, as employed herein, refers to any pathological state involving retinal damage caused by a phenomenon of ischemia and/or excitotoxicity. Non-limiting examples of such diseases include glaucoma, glaucomatous optic neuropathies without hypertonia (such as normal tension glaucoma), age-related macular degeneration, acute or chronic intraocular inflammations (uveitis, uveoretinitis, choroiditis), ischemic or toxic optic neuropathies, endophthalmitis, infectious retinitis, diabetic retinopathy, retinopathy of prematurity, ischemic retinitis proliferans, retinitis pigmentosa, retinopathies associated with a hemoglobinopathy such as sickle cell disease or thalassemia, photodegeneration or else retinal detachment, retinal and choroidal vascular disorders (stenosis, thrombosis, occlusion), retinal and/or choroidal hemorrhage, myopia or else hereditary or acquired retinal degeneration.

Said eye diseases can also arise from the side effects of certain ophthalmic treatments in particular eye surgery, photocoagulation, dynamic phototherapy, transpupillary thermotherapy, intraocular gas injection, administration of certain medicaments such as for example interferons, corticosteroids, anti-angiogenics, vasoconstrictors, chemotherapy or else radiotherapy. To prevent said diseases, the blocker according to the invention can be administered before and/or simultaneously and/or after the therapeutic treatment liable to cause retinal damage.

Some eye diseases involving retinal cell degeneration caused by an ischemic or excitotoxic phenomenon can also originate in arterial, venous or microvascular disorders, hematologic abnormalities or hemorrhages or infarcts.

According to a particular embodiment, the disease associated with retinal ischemia and/or retinal excitotoxicity is selected from the group consisting of glaucoma, glaucomatous optic neuropathies without hypertonia, age-related macular degeneration, diabetic retinopathy, retinopathy of prematurity, ischemic retinitis proliferans and retinitis pigmentosa.

According to another particular embodiment, the disease associated with retinal ischemia and/or retinal excitotoxicity is selected from the group consisting of glaucomatous optic neuropathies without hypertonia, acute or chronic intraocular inflammations (uveitis, uveoretinitis, choroiditis), ischemic or toxic optic neuropathies, endophthalmitis, infectious retinitis, retinopathy of prematurity, retinitis pigmentosa, retinopathies associated with a hemoglobinopathy such as sickle cell disease or thalassemia, photodegeneration or else retinal detachment, retinal and choroidal vascular disorders (stenosis, thrombosis, occlusion), retinal and/or choroidal hemorrhage, or else hereditary or acquired retinal degeneration.

According to another particular embodiment, the disease associated with retinal ischemia and/or retinal excitotoxicity is selected from the group consisting of normal tension glaucoma, uveitis, uveoretinitis, choroiditis, infectious retinitis, retinopathy of prematurity, retinitis pigmentosa, retinopathies associated with sickle cell disease or thalassemia and retinal and/or choroidal hemorrhage.

According to another particular embodiment, the disease associated with retinal ischemia and/or retinal excitotoxicity is myopia.

According to a preferred embodiment, the disease associated with retinal ischemia and/or retinal excitotoxicity is glaucoma or diabetic retinopathy. According to a particularly preferred embodiment, the disease associated with retinal ischemia and/or retinal excitotoxicity is glaucoma.

According to a particular embodiment, the disease associated with retinal ischemia and/or retinal excitotoxicity is diabetic retinopathy and the blocker of ion channels regulated by the SUR subunit is selected from the group consisting of glibenclamide, acetohexamide, carbutamide, glibornuride, chlorpropamide, tolbutamide, tolazamide, glipizide, gliquidone, glyclopyramide, glisoxepide, glimepiride, repaglinide, nateglinide and mitiglinide, and any combination thereof. Preferably, the blocker of ion channels regulated by the SUR subunit is glibenclamide.

The subject to be treated, or patient, is an animal, preferably a mammal. According to one embodiment, the subject to be treated is an animal selected from the group consisting of a dog, a cat, a horse, a cow, a sheep, a pig and a non-human primate. According to a preferred embodiment, the subject to be treated is a human being selected from the group consisting of an infant, a child or an adult.

The blocker according to the invention is administered to the patient in the form of a pharmaceutical composition comprising at least one blocker of ion channels regulated by the SUR subunit and a pharmaceutically acceptable vehicle and/or excipient. Pharmaceutically acceptable vehicles and excipients that can be used are well known to one skilled in the art (Remington's Pharmaceutical Sciences, 18^{th} edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3^{rd} edition, A. Kibbe, Ed., Pharmaceutical Press [2000]). The pharmaceutical composition comprising the blocker of ion channels regulated by the SUR subunit can be formulated as tablets, capsules, soft capsules, granulates, suspensions, emulsions, solutions, polymers, nanoparticles, microspheres, suppositories, lavages, gels, pastes, ointments, creams, medical plasters, potions, injectables, implants, sprays or aerosols.

The blocker of ion channels regulated by the SUR subunit used according to the present invention can be administered by the oral, sublingual, cutaneous, subcutaneous, intramuscular, intravenous, parenteral, topical, local, intratracheal, intranasal, transdermal, rectal, intravitreal, intracameral, subretinal, suprachoroidal, intraocular, periocular, subconjunctival or intra-auricular route. Preferably, the blocker of ion channels regulated by the SUR subunit is administered by the intravitreal, subconjunctival or oral route or by topical instillation. The compound can also be used as irrigation agent during vitreoretinal surgery or as adjuvant in intraocular surgery. It can then eventually be co-administered with a degradable or non-degradable internal tamponade agent.

The blocker according to the invention is administered to the patient at a therapeutically effective dose. The term “therapeutically effective dose” such as employed herein is understood to mean the amount of blocker of ion channels regulated by the SUR subunit that is necessary to achieve a therapeutic or preventive activity on an eye disease associated with retinal ischemia and/or retinal excitotoxicity. The amount of blocker of ion channels regulated by the SUR subunit to be administered and the duration of treatment are evaluated by one of skill in the art according to the physiological condition of the subject to be treated, the pathology to be treated or prevented, the blocker that is chosen and the route of administration used. The blocker according to the invention can be administered as a single dose or as multiple doses.

According to one embodiment of the invention, the blocker is administered to the patient by the intravitreal route in the form of a pharmaceutical composition comprising between 0.01 pg and 100 μg of blocker per unit dose, preferably between 0.01 ng and 1000 ng of blocker, and most preferably between 0.01 ng and 100 ng of blocker.

According to another embodiment of the invention, the blocker is administered to the patient by the oral route in the form of a pharmaceutical composition comprising between 0.001 mg and 150 mg of blocker per kilo of body weight per unit dose, preferably between 0.01 mg and 150 mg of blocker per kilo of body weight, and in a particularly preferred manner between 5 and 15 mg of blocker per kilo of body weight. According to yet another embodiment, the blocker is administered to the patient by the subconjunctival route in the form of a pharmaceutical composition comprising between 0.1 pg and 1000 ng of blocker per unit dose, preferably between 0.1 ng and 100 ng of blocker, and in a particularly preferred manner between 1 ng and 100 ng of blocker.

According to a particular embodiment, the blocker is administered to the patient by the intravitreal route in the form of a pharmaceutical composition comprising between 0.1 pg and 1 pg of blocker per unit dose, from one to four times a month. Preferably, the compound is formulated in a prolonged release system allowing it to be injected every four to eight weeks. In a particularly preferred manner, the compound is formulated in a prolonged release system allowing it to be injected two to four times a year.

The present invention also relates to the use of an blocker of ion channels regulated by the SUR subunit for producing a medicament intended for the treatment and/or prevention of an eye disease associated with retinal ischemia and/or retinal excitotoxicity.

The present invention further relates to a method for treating and/or preventing an eye disease associated with retinal ischemia and/or retinal excitotoxicity in a subject, said method comprising administering a therapeutically effective dose of a blocker of ion channels regulated by the SUR subunit to said subject. According to one embodiment, the subject to be treated is an animal selected from the group consisting of a dog, a cat, a horse, a cow, a sheep, a pig and a non-human primate. According to a preferred embodiment, the subject to be treated is a human being.

All the references cited in this description are incorporated by reference in the present application. Other features and advantages of the invention will become clearer in the following examples which are given for purposes of illustration and not by way of limitation.

### EXAMPLES

### Materials and Methods

Model of NMDA-Induced Retinal Degeneration in the Rat

To induce retinal neurodegeneration, the inventors injected N-methyl-D-aspartate (NMDA) into the vitreous body of rats, a recognized model of retinal excitotoxicity in which NMDA, a synthetic glutamate analog, promotes cell death (Crisanti et al., 2006). Present in excess amounts, NMDA hyperactivates neuronal excitatory receptors (NMDA receptors) which become permeable to calcium. Injection of NMDA results in an increase in intracellular calcium levels. Intracellular calcium activates enzymes (phospholipase C, endonucleases, proteases) which degrade cell structures and lead to apoptosis.

Animals

Eight-week-old male Wistar rats weighing 320-350 g were used. Rats were maintained in standard animal housing with a 12 hr day/12 hr night cycle at a temperature of 21° C.+1° C. and 55±5% humidity.

Anesthesia

Rats were anesthetized by intramuscular injection of sodium pentobarbital (30 mg/kg, CEVA Santé Animate). Tetracaine© was used as local anesthesia for the eyes (collyrium, 1×, 0.4 mL unidose, TVM Laboratories, Lempdes, France).

Frozen Sections

Dissected rat eyes were placed in “cubic” molds filled with specimen matrix compound (Tissue-Tek© OCT—Optimal Cutting Temperature). The molds were then plunged in liquid nitrogen at −80° C. to freeze the eyes. The tissue blocks obtained with these molds were cubic-shaped, allowing them to be sectioned cleanly, avoiding inadvertent movement of the globes which could alter the arrangement of the ocular tissues.

10 μm thick sections were prepared at −20° C. and mounted on pre-gelatinized slides to allow adherence.

TUNEL Method

(Tunel=TdT mediated dUTP-biotin Nick End Labeling; TdT=Terminal deoxynucleotidyl Transferase)

This method is used to detect apoptotic cells. It is based on the fact that apoptosis is accompanied by DNA fragmentation. The TdT enzyme adds a labeled nucleotide to the 3′-OH ends of the fragmented DNA. The signal is then amplified by a peroxidase.

Retinal sections were permeabilized by incubation in 0.3% Triton ×100 for 20 minutes at room temperature and then incubated for 1 hour at 37° C. with 50 μL of TUNEL reaction mixture per slide (mixture obtained with 40 μL TUNEL enzyme and 360 μL TUNEL label, solution containing FITC-conjugated nucleotides).

Sections were washed five times in 1× PBS and once in distilled water, mounted on slides and examined under a fluorescence microscope (for TUNEL-FITC: excitation wavelength 488 nm and emission wavelength 520 nm; for DAPI: excitation wavelength 372 nm and emission wavelength 456 nm).

The amount of cell death by apoptosis and the type of cells concerned were evaluated by the number and intraretinal location of TUNEL-positive nuclei. For each eye, four complete retinal sections taken from the same eye sectors were used for apoptotic cell counts. The total number of apoptotic cells per retina was calculated with reference to the number of fields counted (therefore to retinal length which varies from one area of the retina to another). The mean of these field counts was calculated for an entire retinal section (composed of 7 to 14 fields) and these means, calculated for the different treatment groups, were compared by a nonparametric Mann-Whitney test with significance at p<0.05.

### Example 1

### Effect of Glibenclamide on NMDA-Induced Retinal Degeneration

Experimental Protocol

Dimethylsulfoxide (DMSO) was needed to solubilize the glibenclamide. To reduce the number of intravitreal injections, NMDA and glibenclamide (5 g, MPBIO® Europe, Illkirch, France) previously dissolved in DMSO were co-injected in a same solution after checking that the mixture did not precipitate at the concentrations used. DMSO was used at the lowest concentration required to dissolve glibenclamide (260 mM).

Three glibenclamide solutions containing 1, 10 or 100 ng glibenclamide per 3 μL of solution were prepared. For this, a stock solution containing 9 mg of glibenclamide powder dissolved in 900 μL DMSO (10 μg/μL glibenclamide concentration) was diluted in 1× PBS to obtain the desired concentrations.

As per the experimental protocol described below, the test groups received a co-injection of glibenclamide and NMDA prepared by mixing 3 μL of glibenclamide solution (comprising 1, 10 or 100 ng glibenclamide) and 3 μL of 90 nM NMDA (for final concentration of 45 nM NMDA in the injected solution).

The experiment comprised six groups of rats as follows:

Group 1: (n=2, 4 eyes), injection of 6 μL of solution containing 45 nM NMDA, 1 ng glibenclamide and 260 mM DMSO.

Group 2: (n=2, 4 eyes), injection of 6 μL of solution containing 45 nM NMDA, 10 ng glibenclamide and 260 mM DMSO.

Group 3: (n=2, 4 eyes), injection of 6 μL of solution containing 45 nM NMDA, 100 ng glibenclamide and 260 mM DMSO.

Group 4: (n=2, 4 eyes), injection of 6 μL of solution containing only 45 nM NMDA in 1× PBS buffer (control group).

Group 5: (n=2, 4 eyes), injection of 6 μL of solution containing only 260 mM DMSO in 1× PBS buffer (control group).

Group 6: (n=2, 4 eyes), injection of 6 μL of solution containing 45 nM NMDA and 260 mM DMSO (control group),

24 hours after the intravitreal injections, the rats were euthanized by CO₂ inhalation for 1 minute. The eyes were immediately dissected and briefly cleaned with 1× PBS before being pierced with a 20 G needle to inject paraformaldehyde. Eyes were fixed overnight at 4° C. in 4% paraformaldehyde diluted in 1× PBS. After fixation, the eyes were immersed in a solution containing 20% sucrose diluted in 1× PBS until they floated (approximately 2 hours). This step was designed to prevent any tissue alterations caused by quick-freezing. Eyes were then embedded in OCT, cryostat-sectioned (10 μm thick) and labeled with the TUNEL method to detect apoptotic cell death.

Methods Validation

Negative Control and Positive Control in the Experiment

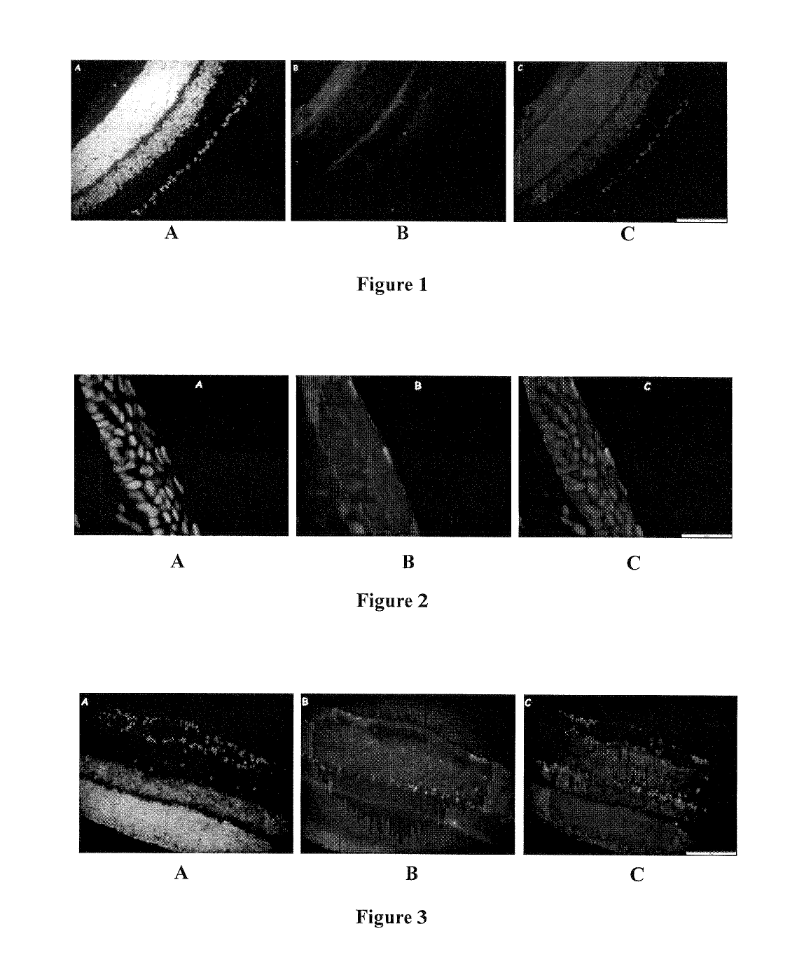

The negative control was a retinal slide from the “NMDA+DMSO” control group (group 6) in which TUNEL enzyme was not added when the slides were prepared with the TUNEL method (only the TUNEL label solution was added). The absence of TUNEL-positive (DAPI-labeled) nuclei, and therefore of apoptotic cells, validates the experiments (FIG. 1).

The positive control verified that the TUNEL method worked correctly. Superficial corneal epithelial cells from each section, among which apoptotic cells (desquamating cells) are always present, were used as positive control. The positive TUNEL labeling (FIG. 2B) correlates well with the corneal epithelium nuclei (FIG. 2A), therefore indicating that the apoptotic cell nuclei were indeed labeled. The TUNEL method was validated in this manner.

NMDA-Induced Retinal Cell Degeneration

24 hours after intravitreal NMDA injection (group 4), numerous TUNEL-positive cells were visible in the internal nuclear layer (FIG. 3B and C). Since NMDA damages the retina from the surface to the interior, this finding demonstrates the advanced stage of retinal degeneration induced by NMDA.

Effect of DMSO on Retinal Cells (Healthy or Degenerated)

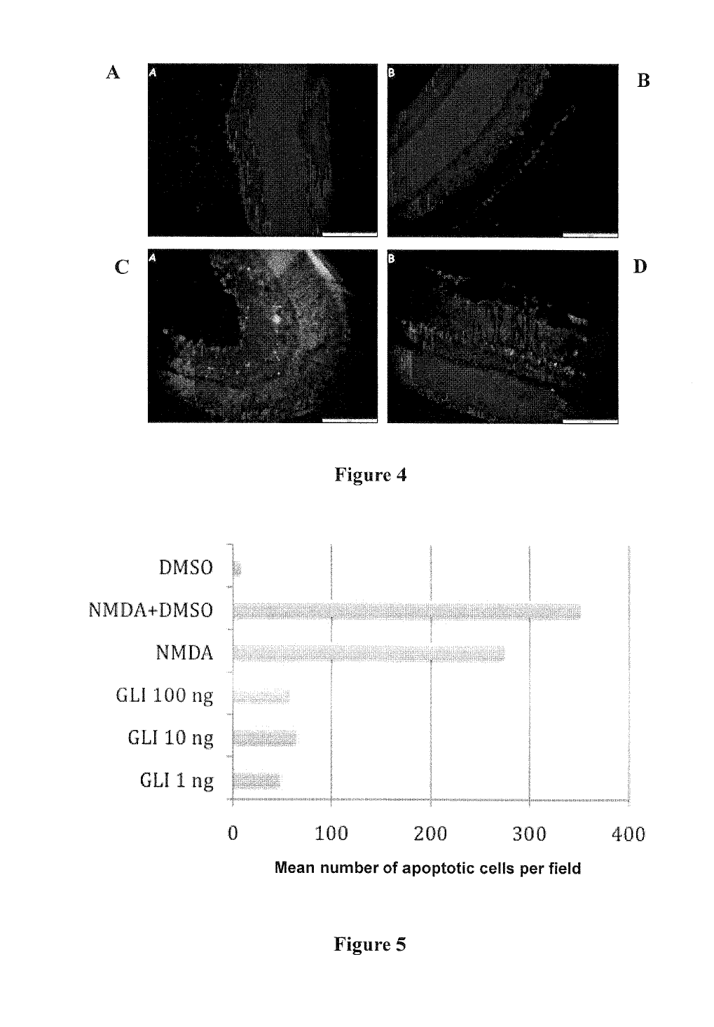

TUNEL labeling after DMSO treatment alone (group 5) was similar to the labeling pattern of negative controls, confirming that DMSO was not toxic to healthy retinal cells at the concentration used for the experiment (FIG. 4A and B).

TUNEL labeling after treatment with DMSO and NMDA (group 6) and after NMDA alone (group 4) showed an equivalent quantity of apoptotic cells (FIG. 4C and D).

These results confirm the absence of either a toxic or a beneficial effect of DMSO on NMDA-induced degeneration.

Results

The retinal sections obtained from the six groups of rats and labeled with DAPI and TUNEL were examined under a microscope and the mean number of apoptotic cells (i.e., TUNEL-positive cells) per field was determined for each group. These results are shown in the histogram of FIG. 5.

These data illustrate firstly the absence of either a toxic or a beneficial effect of DMSO on NMDA-induced degeneration of the retinal cells.

The quantity of apoptotic cells observed in the groups co-injected with glibenclamide and NMDA was 82 to 86% lower than the quantity of apoptotic cells observed in the groups injected with NMDA in the presence of DMSO.

These findings therefore demonstrate that injection of glibenclamide significantly decreased NMDA-induced retinal cell degeneration.

### Example 2

### Effect of Different Routes of Administration of Glibenclamide on Retinal Degeneration

Experimental Protocol

Eight groups of 2 rats were used. Both eyes of each animal received identical treatment. Three routes of administration of glibenclamide were tested: intravitreal route (1 ng or 0.01 ng injected in each eye), oral route (10 mg/kg body weight) and subconjunctival route (1 ng glibenclamide injected in each eye).

The glibenclamide stock solution for the intravitreal and subconjunctival injections was obtained by diluting 4.5 mg glibenclamide in 300 μL of pure DMSO. This solution was then frozen. The glibenclamide solutions at different concentrations used for the intravitreal and subconjunctival injections were obtained by dilution in BSS® buffer (Alcon) immediately before use.

The glibenclamide solutions for oral administration (10 mg/kg) were obtained by dissolving 8 mg glibenclamide in 300 μM DMSO and 1030 μL of water to give a 6 mg/ml glibenclamide solution, which was administered through a syringe.

NMDA was diluted in water and buffered with TRIS pH 7.

The experimental protocol comprised eight groups of rats as follows:

Group 1: (n=2, 4 eyes), intravitreal injection of 6 μL of solution containing 45 nM NMDA and 1 ng glibenclamide.

Group 2: (n=2, 4 eyes), intravitreal injection of 6 μL of solution containing 45 nM NMDA and 0.01 ng glibenclamide.

Group 3: (n=2, 4 eyes), intravitreal injection of 6 μL of solution containing 45 nM NMDA and 260 mM DMSO.

Group 4: (n=2, 4 eyes), oral administration of glibenclamide 10 mg/kg body weight and simultaneous intravitreal injection of 6 μL of 45 nM NMDA. A 7% glucose solution was made available ad libitum to avoid possible hypoglycemia.

Group 5: (n=2, 4 eyes), oral administration of 260 mM DMSO and simultaneous intravitreal injection of 6 μL of 45 nM NMDA. A 7% glucose solution was made available ad libitum to avoid possible hypoglycemia.

Group 6: (n=2, 4 eyes), subconjunctival injection of 50 μL of solution containing 1 ng glibenclamide and simultaneous intravitreal injection of 6 μL of 45 nM NMDA.

Group 7: (n=2, 4 eyes), subconjunctival injection of 50 μL of 260 mM DMSO and simultaneous intravitreal injection of 6 μL of 45 nM NMDA.

Group 8: (n=2, 4 eyes), no treatment.

18 hours after NMDA and glibenclamide administration, rats were euthanized and the eyes were immediately dissected, fixed in 4% paraformaldehyde and 10% sucrose for 4 hours, then immersed overnight at 4° C. in 15% sucrose in phosphate buffer.

Eyes were then embedded in OCT and cryostat-sectioned (10 μm thick). Sections were labeled by the TUNEL method to detect apoptotic cell death.

Results

The methods used in this example were first validated according to the protocol described in example 1 (“Methods validation”).

Intravitreal Injection of Glibenclamide

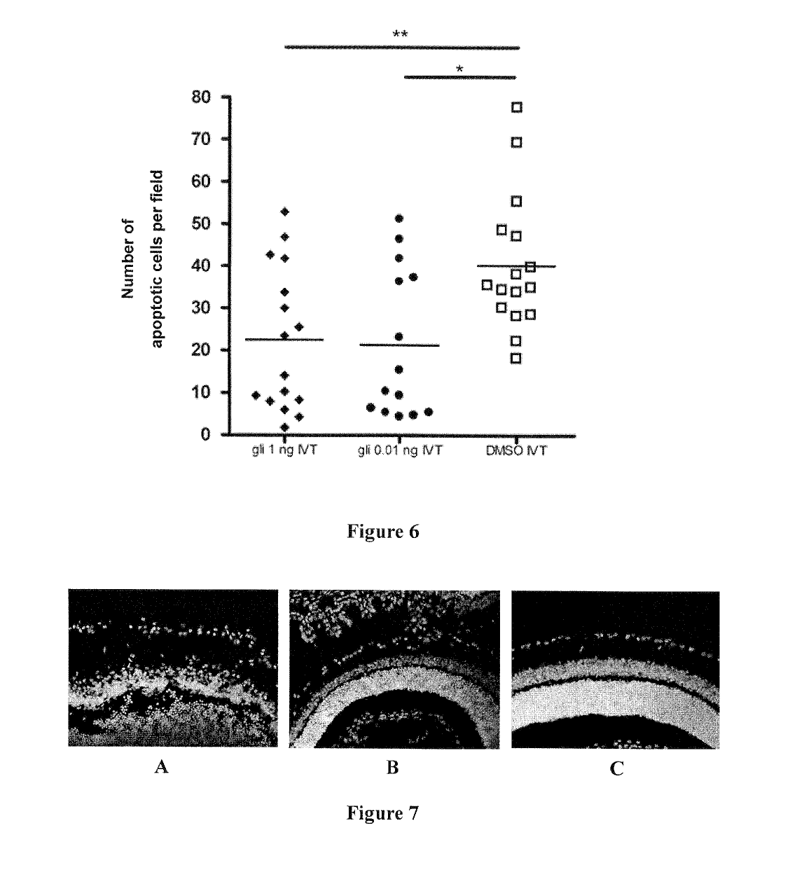

Intravitreal injection of 1 ng (group 1) or 0.01 ng glibenclamide (group 2) led to a significant decrease in the apoptotic cell count in retinas exposed to NMDA-induced degeneration (FIG. 6). Labeling of these retinas also revealed that injection of 1 or 0.01 ng glibenclamide perfectly conserved the organization of the retinal cell layers (FIG. 7B and C) as opposed to what was observed in the degenerated retinas (FIG. 7A).

These findings demonstrate that glibenclamide significantly reduced NMDA-induced retinal degeneration and destructuration of the retinal layers, and this, even at a very low dose.

Oral Administration of Glibenclamide

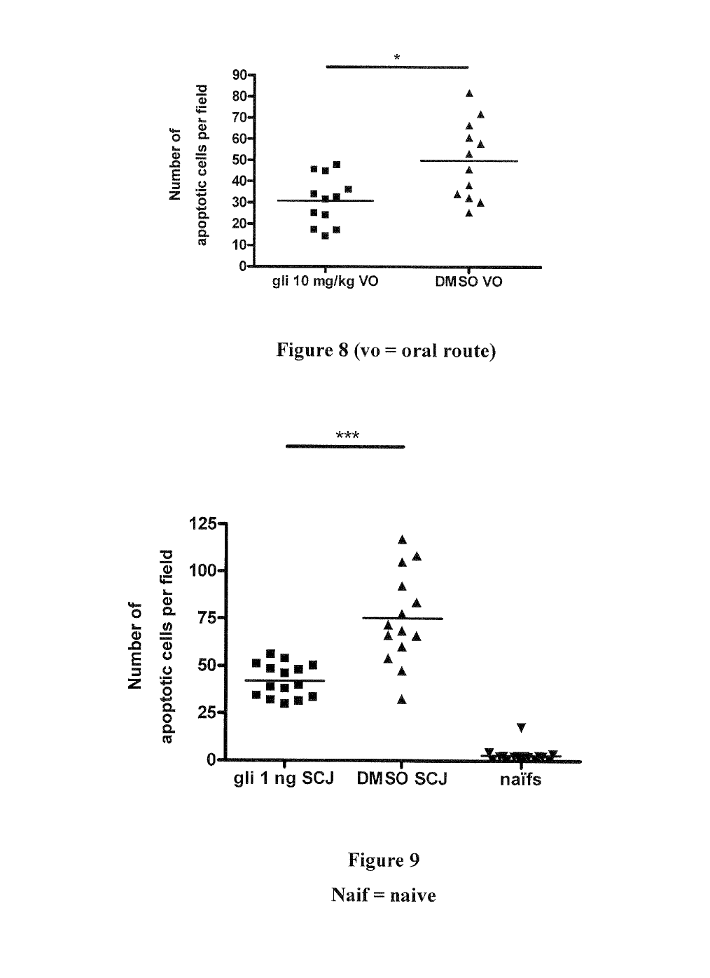

Oral administration of glibenclamide and intravitreal injection of NMDA were carried out the same day (group 4). The results obtained for rats in groups 4 and 5 are shown in FIG. 8.

These results show that oral administration of glibenclamide significantly reduced NMDA-induced retinal cell degeneration.

Subconjunctival Administration of Glibenclamide

Subconjunctival administration of glibenclamide and intravitreal injection of NMDA were carried out the same day (group 6). The results obtained for rats in groups 6 and 7 are shown in FIG. 9.

It can be seen that subconjunctival administration of glibenclamide significantly reduced NMDA-induced retinal cell degeneration.

### Example 3

### Preventive Effect of Glibenclamide on NMDA-Induced Retinal Degeneration

Experimental Protocol

Three groups of 2 rats were used. Both eyes of each animal received identical treatment.

The preventive effect of glibenclamide was evaluated by injecting into the vitreous body of each eye 100 ng or 100 μg glibenclamide diluted in BSS® buffer, 5 days before intravitreal injection of NMDA. Glibenclamide was diluted in BSS buffer and not in DMSO so as to obtain a prolonged-effect suspension, resulting from the low solubility of glibenclamide.

Administration of 100 μg glibenclamide allowed a test of the potential toxicity of a high dose.

The glibenclamide stock solution for intravitreal injections was obtained by diluting 4.5 mg glibenclamide in 300 μL of pure DMSO. This solution was then frozen. The glibenclamide solutions at different concentrations used for the intravitreal injections were obtained by dilution in BSS® buffer (Alcon) immediately before use.

NMDA was diluted in water and buffered with IRIS pH 7 to obtain a concentration of 90 nM.

The experimental protocol comprised three groups of rats as follows:

Group 1: (n=2, 4 eyes), D-5: intravitreal injection of 3 μL of solution containing 100 glibenclamide in BSS® buffer; D0: intravitreal injection of 3 μL of 90 nM NMDA

Group 2: (n=2, 4 eyes), intravitreal injection of 3 μL of solution containing 100 ng glibenclamide in BSS® buffer; D0: intravitreal injection of 3 μL of 90 nM NMDA

Group 3: (n=2, 4 eyes), D-5: intravitreal injection of 3 μL of BSS buffer; D0: intravitreal injection of 3 μL of 90 nM NMDA.

18 hours after NMDA and glibenclamide administration, rats were euthanized and the eyes were dissected immediately, fixed in 4% paraformaldehyde and 10% sucrose for 4 hours, then immersed overnight at 4° C. in 15% sucrose in phosphate buffer.

Eyes were then embedded in OCT and cryostat-sectioned (10 μm thick). The sections were labeled by the TUNEL method to detect apoptotic cell death.

Results

The methods used in this example were first validated according to the protocol described in example 1 (“Methods validation”).

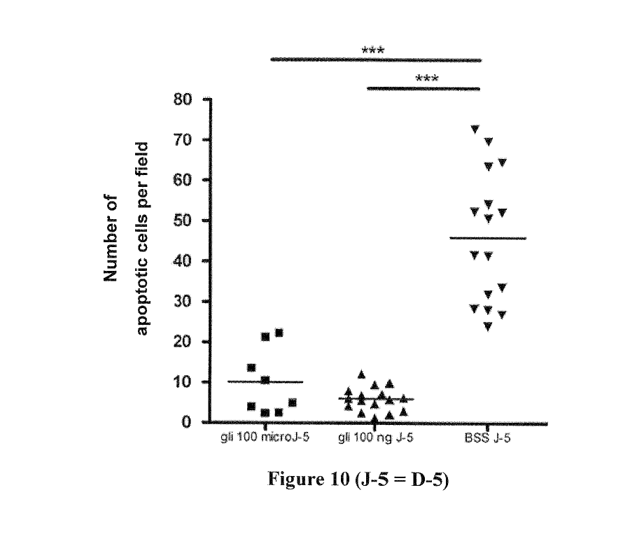

Intravitreal injection of 100 μg (group 1) or 100 ng (group 2) glibenclamide 5 days before intravitreal injection of NMDA significantly protected retinal cells from NMDA-induced degeneration (FIG. 10).

In addition, the protective effect of glibenclamide administered at a high dose (100 μg) demonstrates the total absence of toxicity of this molecule on retinal cells.

### Conclusion

NMDA induces neurodegeneration of retinal cells 18 to 24 hours after injection. NMDA acts first on the ganglion cell layer, which is the most superficial layer and in contact with the vitreous body, then on deeper layers before reaching the photoreceptors. Both 18 hours and 24 hours after injection, NMDA had already damaged the ganglion cell layer and induced apoptosis of the cells of the internal nuclear layer.

Through these different examples the inventors have demonstrated that administration of glibenclamide by the intravitreal, oral or subconjunctival route led to a significant reduction in retinal cell apoptosis induced by NMDA.

The inventors have also demonstrated that preventive administration of glibenclamide a few days before NMDA injection efficiently prevented retinal cell degeneration.

### BIBLIOGRAPHIC REFERENCES

Celebi S, Dilsiz N, Yilmaz T, Kukner A S. Effects of minelatonin, vitamin E and octreotide on lipid peroxidation during ischemia-reperfusion in the guinea pig 15 retina. Eur J Ophthalmol. 2002 March-April;12(2):77-83.

Chao H M, Osborne N N. Topically applied clonidine protects the rat retina from ischaemia/reperfusion by stimulating alpha(2)-adrenoceptors and not by an action on imidazoline receptors. Brain Res. 2001 Jun. 15;904(1):126-36.

Crisanti P, Laplace O, Lecain E, Jonet L, Jeanny J C, Omri B. The role of PKCζ, in NMDA-induced retinal ganglion cell death: Prevention by aspirin. Apoptosis 2006; 11:983-991.

Donello J E, Padillo E U, Webster M L, Wheeler L A, Gil D W. alpha(2)Adrenoceptor agonists inhibit vitreal glutamate and aspartate accumulation and preserve retinal function after transient ischemia. J Pharmacol Exp Ther. 2001 January;296(1):216-23.

Fontaine V, Mohand-Said S, Hanoteau N, Fuchs C, Pfizenmaier K, Eisel U. Neurodegenerative and neuroprotective effects of Tumor Necrosis Factor (TNF) in retinal ischemia: opposite roles of TNF receptor 1 and TNF receptor 2. J Neurosci. 2002 Apr. 1;22(7).

Lam T T, Siew E, Chu R, Tso MOM. Ameliorative effect of MK801 on retinal ischemia. J Ocular Pharmacol Ther. 1997;13:129-37.

Liss B, Roeper J. Molecular physiology of neuronal K-ATP channels. Mol Membr Biol. 2001;18:117-127.

Nistico R, Piccirilli S, Sebastianelli L, Nistico G, Bernardi G, Mercuri N B. The blockade of K (+)-ATP channels has neuroprotective effects in an in vitro model of brain ischemia. Int Rev Neurobiol. 2007; 82:383-95.

Osborne N N, Schwarz M, Pergande G. Protection of rabbit retina from ischemic injury by flupirtine. Invest Ophthalmol Vis Sci. 1996;37:274-80.

Pitlik S, Manor R S, Lipshitz I, Perry G, Rosenfeld J. Transient retinal ischaemia induced by nifedipine. Br Med J (Clin Res Ed). 1983 Dec. 17;287(6408):1845-6.

Sherry D M, Townes-Anderson E. Rapid glutamatergic alterations in the neural retina induced by retinal detachment. Invest Ophthalmol Vis Sci. 2000 August;41(9):2779-90.

Simard J M, Chen M, Tarasov K V, Bhatta S, Ivanova S, Melnitchenko L, Tsymbalyuk N, West G A, Gerzanich V. Newly expressed SUR1-regulated NC(Ca-ATP) channel mediates cerebral edema after ischemic stroke. Nat Med. 2006 April;12(4):433-40.

Simard J M, Woo S K, Bhatta S, Gerzanich V. Drug acting on SUR1 to treat CNS ischemia and trauma. Curr Opin Pharmacol. 2008 February;8(1):42-9.

Szabo M E, Haines D, Garay E, Chiavaroli C, Farine J C, Hannaert P, Berta A, Garay RPL. Antioxidant properties of calcium dobesilate in ischemic/reperfused diabetic rat retina. Eur J Pharmacol. 2001 Oct. 5;428(2):277-86.

Tsukahara Y, Blair N P, Spellman Eappen D C, et al. Ketamine suppresses ischemic injury in the rabbit retina. Invest Ophthalmol Vis Sci. 1992;33:1822-5.

Vorwerk C K, Lipton S A, Zurakowski D, et al. Chronic low-dose glutamate is toxic to retinal ganglion cells. Toxicity blocked by memantine. Invest Ophthalmol Vis Sci. 1996;37:1619-24.

Yoon Y H, Marmor M F. Dextromethorphan protects retina against ischemic injury in vivo. Arch Ophthalmol. 1989;107:409-11.

1-10. (canceled)

11. A method of treating a disease associated with retinal ischemia and/or retinal excitotoxicity in a subject, said method comprising administering an effective therapeutic dose of a blocker of ion channels regulated by the SUR subunit to said subject.

12. The method according to claim 11, wherein said blocker is selected from the group consisting of a sulfonylurea and a metglitinide, and a combination thereof.

13. The method according to claim 12, wherein said blocker is selected from the group consisting of glibenclamide, acvetohexamide, carbutamide, glibornuride, chlorpropamide, tolbutamide, tolazamide, glipizide, gliclazide, gliquidone, glyclopyramide, glisoxepide, glimepiride, repaglinide, nateglinide and mitiglinide, and any combination thereof

14. The method according to claim 13, wherein said blocker is glibenclamide.

15. The method according to claim 11, wherein said blocker is administered in combination with another active substance, simultaneously or sequentially.

16. The method according to claim 11, wherein the disease associated with retinal ischemia and/or retinal excitotoxicity is selected from the group consisting of glaucoma, glaucomatous optic neuropathies without hypertonia, age-related macular degeneration, acute or chronic intraocular inflammations, ischemic or toxic optic neuropathies, endophthalmitis, infectious retinitis, diabetic retinopathy, retinopathy of prematurity, ischemic retinitis proliferans, retinitis pigmentosa, retinopathies associated with a hemoglobinopathy, photodegeneration, retinal detachment, retinal and choroidal vascular disorders, retinal and/or choroidal hemorrhage, myopia and hererditary or acquired retinal degeneration.

17. The method according to claim 16, wherein the disease associated with retinal ischemia and/or retinal excitotoxicity is selected from the group consisting of glaucoma, age-related macular degeneration, diabetic retinopathy, retinopathy of prematurity, ischemic retinitis proliferans and retinitis pigmentosa.

18. The method according to claim 11, wherein the subject to be treated is a human being selected from the group consisting of an infant, a child and an adult.

19. The method according to claim 11, wherein the subject to be treated is an animal selected from the group consisting of a dog, a cat, a horse, a cow, a sheep, a pig and a non-human primate.

20. The method according to claim 11, wherein said blocker is administered by the intravitreal route, by the subconjunctival route, by the oral route, by topical instillation, by the periocular and intraocular route or by the parenteral route.

21. The method according to claim 16, wherein said acute or chronic intraocular inflammations are selected from uveitis, uveoretinitis or choroiditis.

22. The method according to claim 16, wherein said retinal and choroidal vascular disorders are selected from vascular occlusions, stenosis or thrombosis.
